(19) United States
(12) Patent Application Publication
(10) Pub. No.: US 2017/0014650 A1
(43) Pub. Date: Jan. 19, 2017

(54) ULTRASONIC TREATMENT APPARATUS

(71) Applicant: OLYMPUS CORPORATION, Tokyo (JP)

(72) Inventor: Kei UEKI, Tokyo (JP)

(73) Assignee: OLYMPUS CORPORATION, Tokyo (JP)

(21) Appl. No.: 15/277,229

(22) Filed: Sep. 27, 2016

Related U.S. Application Data
(63) Continuation of application No. PCT/JP2015/054709, filed on Feb. 20, 2015.

(30) Foreign Application Priority Data
Apr. 17, 2014 (JP) 2014-085458

(51) Int. Cl. A61N 7/00

(52) U.S. Cl. CPC A61N 7/00

(57) ABSTRACT

This ultrasonic treatment apparatus treats a treatment target organ, suppressing raise of the temperature at a surface of the treatment target organ, without making the apparatus large. This ultrasonic treatment apparatus includes an ultrasonic element which faces a surface of a treatment target organ through an acoustic propagation medium and which generates ultrasonic wave converged on a depth position of the treatment target organ, and a controller for adjusting the ultrasonic wave irradiated to the treatment target organ from the ultrasonic element, wherein the controller changes intensity of the ultrasonic wave which is irradiated into an area of the surface so that heat which is generated when the ultrasonic wave radiated by the ultrasonic element passes through the surface and which remains at a vicinity of the surface is kept equal to or less than a predetermined threshold value.

### CROSS-REFERENCE TO RELATED APPLICATIONS

This application is a Continuation Application of International Application No. PCT/JP2015/054709 filed on Feb. 20, 2015, which claims priority to Japanese Application No. 2014-085458 filed on Apr. 17, 2014. The contents of International Application No. PCT/JP2015/054709 and Japanese application No. 2014-085458 are incorporated herein by reference in its entirety.

### TECHNICAL FIELD

The present invention relates to an ultrasonic treatment apparatus.

### BACKGROUND ART

Conventionally, an acoustic propagation medium is filled between the surface of the treatment target organ in the body and the ultrasonic element which faces the surface, and when therapeutic ultrasonic wave is generated from the ultrasonic element, the ultrasonic treatment device treats the treatment target organ while suppressing the temperature rise at the surface by cooling the acoustic propagation medium. (See PTL 1, for example.)

### CITATION LIST

### Patent Literature

{PTL 1} Japanese Unexamined Patent Application, Publication No. H06-217989

### SUMMARY OF INVENTION

An aspect of the present invention provides an ultrasonic treatment apparatus comprising: an ultrasonic element which faces a surface of a treatment target organ through an acoustic propagation medium and which generates ultrasonic wave so that the ultrasonic wave is converged on a depth position of the treatment target organ; and a controller for adjusting the ultrasonic wave irradiated to the treatment target organ from the ultrasonic element, wherein the controller is configured to change intensity of the ultrasonic wave which is irradiated into an area of the surface so that heat which is generated when the ultrasonic wave radiated by the ultrasonic element passes through the surface and which remains at a vicinity of the surface is kept equal to or less than a predetermined threshold value, and the controller performs the intensity change as time passes.

### BRIEF DESCRIPTION OF DRAWINGS

FIG. 1 is a drawing showing an entire structure of an ultrasonic treatment apparatus according to a first embodiment of the present invention.

FIG. 2 is a diagram showing an example of waveform of the ultrasonic wave radiated by the ultrasonic treatment apparatus shown in FIG. 1.

FIG. 3A shows heat generation at a portion in the depth direction when the ultrasonic wave is radiated by the ultrasonic treatment apparatus shown in FIG. 1.

FIG. 3B shows heat generation at the portion in the depth direction when the ultrasonic wave is radiated by the ultrasonic treatment apparatus shown in FIG. 1.

FIG. 3C shows heat generation at the portion in the depth direction when the ultrasonic wave is radiated by the ultrasonic treatment apparatus shown in FIG. 1.

FIG. 4 is a diagram showing a modified example of the ultrasonic waveform shown in FIG. 2.

FIG. 5A is a diagram showing a modified example of the ultrasonic treatment apparatus shown in FIG. 1 with an ultrasonic element located at one side.

FIG. 5B is a diagram showing the modified example of the ultrasonic treatment apparatus shown in FIG. 1 with the ultrasonic element located at the other side.

FIG. 6A is a diagram showing another modified example of the ultrasonic treatment apparatus shown in FIG. 1, showing a state in which an ultrasonic element located at one side is operated.

FIG. 6B is a diagram showing said modified example of the ultrasonic treatment apparatus shown in FIG. 1, showing a state in which an ultrasonic element located at the other side is operated.

FIG. 7 is a drawing showing an entire structure of an ultrasonic treatment apparatus according to a second embodiment of the present invention.

FIG. 8A shows a position of an ultrasonic element of the ultrasonic treatment apparatus shown in FIG. 7, and shows heat generation at the portion in the depth direction at a started portion when the ultrasonic is located at the position.

FIG. 8B shows a position of an ultrasonic element of the ultrasonic treatment apparatus shown in FIG. 7, and shows heat generation at the portion in the depth direction at the started portion when the ultrasonic is located at the position.

FIG. 8C shows a position of an ultrasonic element of the ultrasonic treatment apparatus shown in FIG. 7, and shows heat generation at the portion in the depth direction at the started portion when the ultrasonic is located at the position.

FIG. 8D shows a position of an ultrasonic element of the ultrasonic treatment apparatus shown in FIG. 7, and shows heat generation at the portion in the depth direction at the started portion when the ultrasonic is located at the position.

FIG. 8E shows a position of an ultrasonic element of the ultrasonic treatment apparatus shown in FIG. 7, and shows heat generation at the portion in the depth direction at the started portion when the ultrasonic is located at the position.

FIG. 9 is a drawing showing an entire structure of a modified example of the ultrasonic treatment apparatus shown in FIG. 7.

FIG. 10 is a drawing showing an example of radiating the ultrasonic by the ultrasonic treatment apparatus shown in FIG. 9.

FIG. 11A shows a position of an ultrasonic element of the ultrasonic treatment apparatus shown in FIG. 7, and shows a state of heat generation when the ultrasonic element is located at the position.

FIG. 11B shows a position of an ultrasonic element of the ultrasonic treatment apparatus shown in FIG. 7, and shows a state of heat generation when the ultrasonic element is located at the position.

FIG. 11C shows a position of an ultrasonic element of the ultrasonic treatment apparatus shown in FIG. 7, and shows a state of heat generation when the ultrasonic element is located at the position.

FIG. 11D shows a position of an ultrasonic element of the ultrasonic treatment apparatus shown in FIG. 7, and shows a state of heat generation when the ultrasonic element is located at the position.

FIG. 11E shows a position of an ultrasonic element of the ultrasonic treatment apparatus shown in FIG. 7, and shows a state of heat generation when the ultrasonic element is located at the position.

FIG. 12 is a drawing showing an entire structure of another modified example of the ultrasonic treatment apparatus shown in FIG. 7.

### DESCRIPTION OF EMBODIMENTS

An ultrasonic treatment apparatus 1 according to a first embodiment of the present invention will be described below with reference to the drawings.

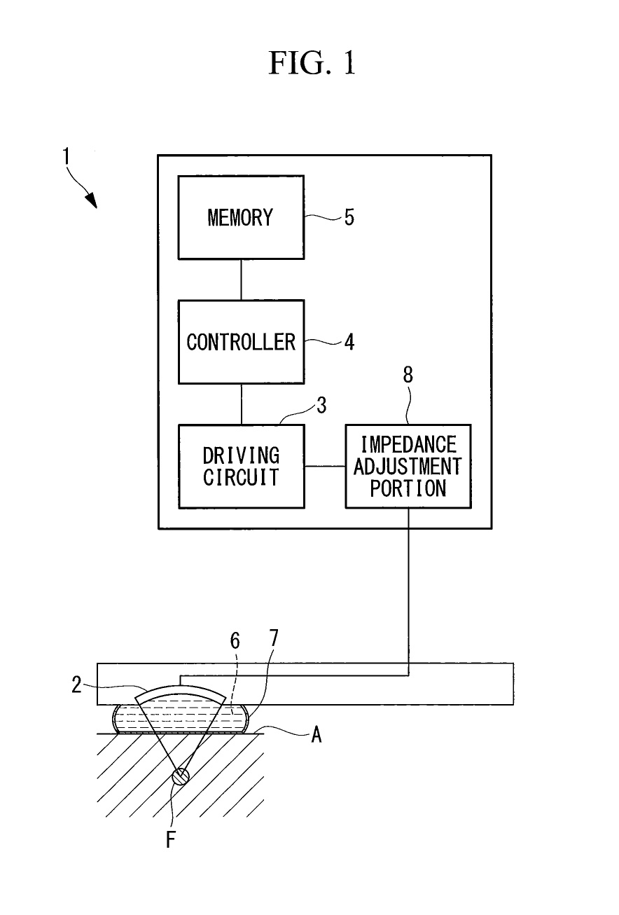

As shown in FIG. 1, the ultrasonic treatment apparatus 1 has an ultrasonic element 2 which generates ultrasonic wave, a driving circuit 3 which drives the ultrasonic element 2, a controller 4 which controls the driving circuit 3, and a memory 5 which stores a condition for radiating ultrasonic wave. Also, a balloon 7 in which an acoustic propagation medium is filled is located between the ultrasonic element 2 and the surface of treatment target organ A, thereby the space between the ultrasonic element 2 and the surface of the treatment target organ A is filled up by the acoustic propagation medium.

The ultrasonic element 2 is a HIFU (High Intensity Focused Ultrasound) element, and has an ultrasonic transducer having a concave surface, and also generates ultrasonic wave so that the ultrasonic wave is converged on a focal point F of the concave surface. The ultrasonic treatment apparatus 1 also has an impedance adjustment portion 8 located between the ultrasonic element 2 and the driving circuit 3 for improving a transmitting efficiency.

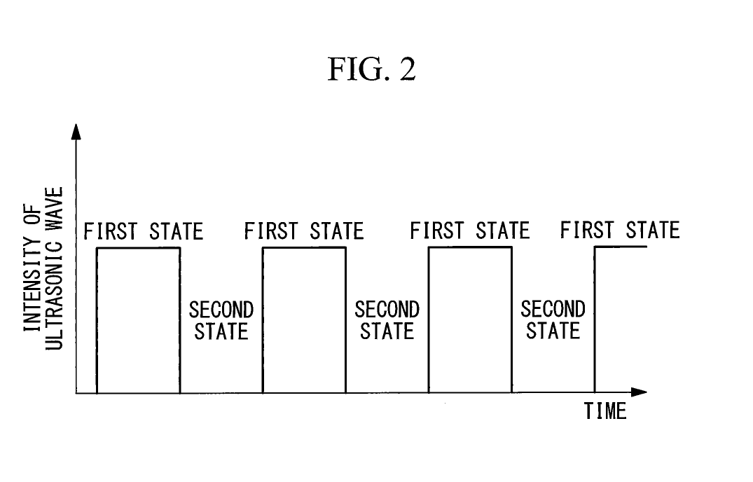

AS shown in FIG. 2, the controller 4 outputs drive command signal to the driving circuit 3 of the ultrasonic element 2 so that a first state being radiating ultrasonic wave by driving the ultrasonic element 2 and a second state being not radiating the ultrasonic wave by stopping driving the ultrasonic element 2 is alternately repeated. For example, the controller 4 is configured with a computer.

The intensity of the ultrasonic wave radiated from the ultrasonic element 2 in the first state, and the duration periods of the first state and the second state are stored in the memory 5 as the condition for radiating ultrasonic wave by the controller 4. The intensity of the ultrasonic wave and the duration of the first state are set to intensity and duration with which allows the temperature of the surface of the treatment target organ A to a temperature reach less than an upper limit temperature that does not cause thermal denaturation. The duration of the second state is set to duration which allows the temperature of the irradiated surface to become a temperature equal to or lower than a predetermined value.

In this embodiment, the intensity of the first state, the radiating time period of the first state, and the non-radiating time period of the second state are constant, respectively.

A function of the ultrasonic treatment apparatus 1 according to this embodiment configured as described above will be described below.

In order to treat the affected area located at a deep part of the treatment target organ A using the ultrasonic treatment apparatus 1 according to this embodiment, the ultrasonic element 2 is located so as to face the affected area located at the deep part of the treatment target organ in a state in which the balloon 7, in which the acoustic propagation medium 6 is filled, is sandwiched between the ultrasonic element 2 and the surface of the treatment target organ A. By this process, the focal point F of the ultrasonic wave from the ultrasonic element 2 matches the affected area in the treatment target organ A.

In this state, the controller 4 outputs the drive command signal in accordance with the condition for radiating ultrasonic wave stored in the memory 5, the driving circuit 3 actuates the ultrasonic element 2, and then the first state which radiates the ultrasonic wave and the non-radiating second state are repeatedly altered as shown in FIG. 2.

In the first state, when the ultrasonic wave is generated by the ultrasonic element 2, the ultrasonic wave generated by the ultrasonic element 2 is propagated through the acoustic propagation medium 6 in the balloon 7 which the ultrasonic element 2 is brought into close contact with, and the ultrasonic wave is irradiated into the treatment target organ A through the surface thereof, and then the ultrasonic wave is converged at the focal point F which matches the affected area.

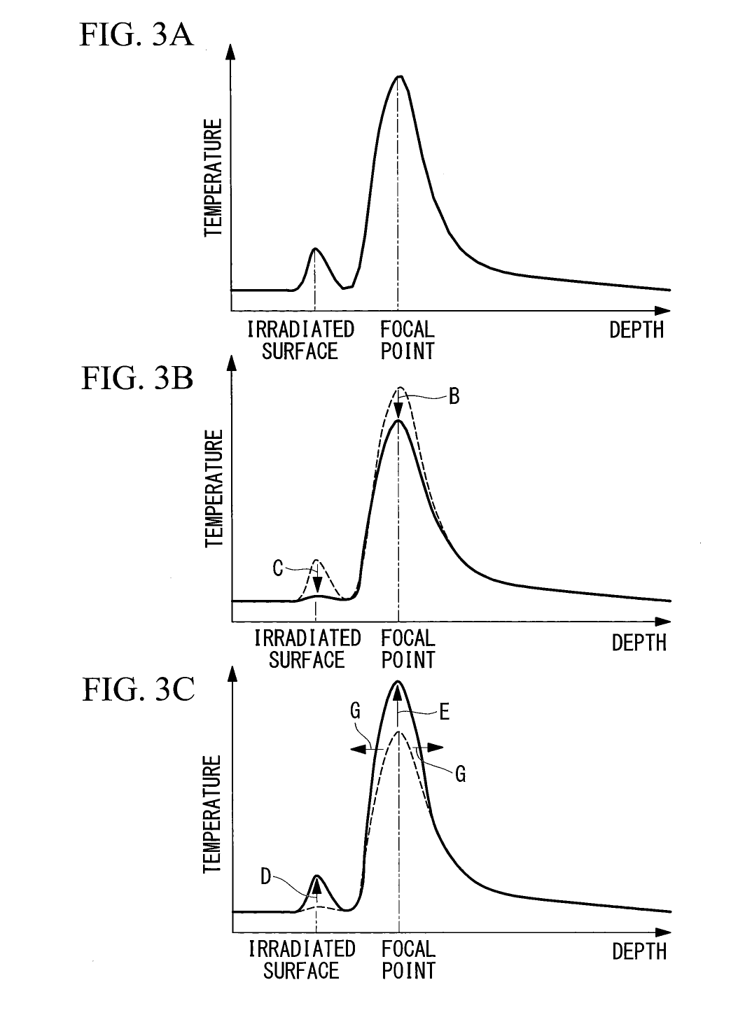

By this process, as shown in FIG. 3A, heat is generated at the focal point F, and the temperature of the focal point F rises. At this stage, heat is generated at a vicinity of the surface (irradiated surface) of the treatment target organ A, wherein the heat is sufficiently smaller than heat generated at the focal point F.

Next, as shown in FIG. 3B, in the second state, when irradiation of the ultrasonic wave is stopped, the heat generation in the treatment target organ A is stopped, and the peak value of the remaining heat decreases by thermal diffusion (arrows B, C). Especially, at the surface portion of the treatment target organ A, heat is transferred to the acoustic propagation medium 6 in the balloon 7 which has contact with the treatment target organ A, and the temperature decreases until the remaining heat becomes substantially zero (arrow C).

In this state, as shown in FIG. 3C, the ultrasonic wave is generated again as the first state, heat is generated at the focal point F and the like, and the temperature rises.

At this stage, since little remaining heat exists at the vicinity of the surface of the treatment target organ A, temperature of the vicinity of the surface of the treatment target organ A is suppressed at a level which is the same as or similar to the first state of the turn (arrow D). In contrast, at the focal point F, the peak of the temperature becomes higher than that of the first state of the first turn when heat is newly generated (arrow E), and the thermal denaturation portion expands toward the depth direction due to the large amount of remaining heat (arrow G).

Then, with repetition of the aforementioned processes, it is possible to make the thermal denaturation portion near the affected area bigger, suppressing the temperature rise of the surface of the treatment target organ A, and also it is possible to efficiently treat the affected area while protecting the portions outside the affected area.

As described above, with the ultrasonic treatment apparatus 1 according to this embodiment, it is possible to reduce the remaining heat which is generated and remained at the vicinity of the surface of the treatment target organ A less than a predetermined value when the ultrasonic wave radiated by the ultrasonic element 2 passes through, by changing the intensity per time of the ultrasonic wave irradiated into the same area on the surface of the treatment target organ A, using alternate repetition between operation and stoppage of the ultrasonic element 2. This configuration leads to an advantage of enabling effective treatment of the affected area where the focal point F is positioned and protection of the portions other than the affected area can be simultaneously achieved.

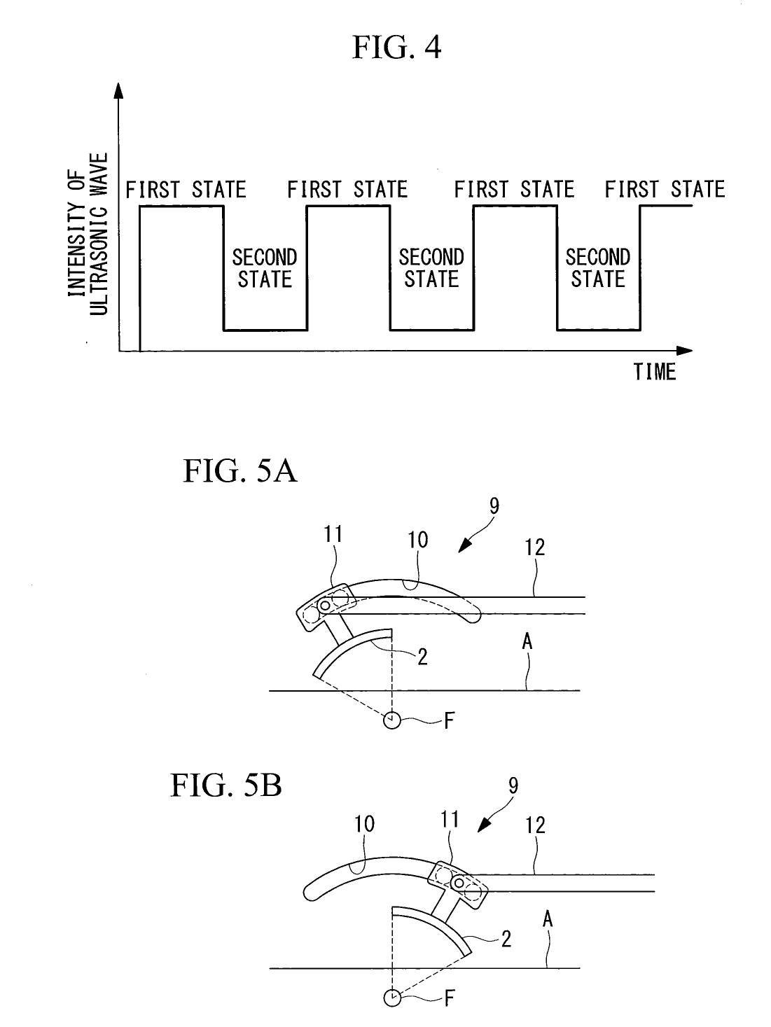

Also, in the ultrasonic treatment apparatus 1 according to this embodiment, the operation of the ultrasonic element 2 is stopped in the second state. In an alternative configuration, as shown in FIG. 4, it is also possible to radiate the ultrasonic wave with a sufficiently low intensity which allows the remaining heat to be reduced. It is also possible to change the duration of radiating the ultrasonic wave and the intensity thereof in the first state, and to change the duration of non-radiation of the ultrasonic wave in the second state.

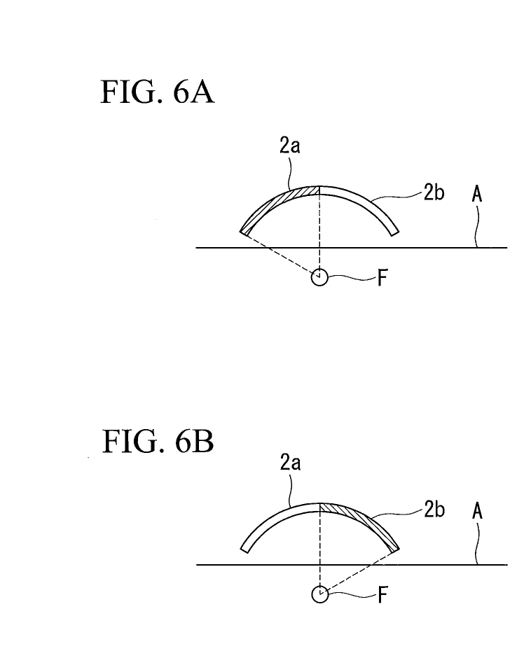

Also, in the ultrasonic treatment apparatus 1 according to this embodiment, by alternatively repeating operation and non-operation of the ultrasonic element 2, heat generation on the surface of the treatment target organ A is suppressed. In an alternative configuration, as shown in FIGS. 5A and 5B, a moving structure (moving unit) 9 for moving the ultrasonic element 2 may be employed.

As the moving structure 9, a configuration in which a slider 11 is moved by a link 12 and along a circular groove 10 can be employed as an example. By aligning the center of the groove 10 with the position of the focal point F of the ultrasonic element 2, it becomes possible to swing the ultrasonic element 2 so that the focal point F is the center of the swing.

By employing this configuration, it becomes possible to treat the affected area using the continuous convergence of the ultrasonic wave on the focal point F by radiating the ultrasonic wave from the ultrasonic element 2 and by swinging the ultrasonic element 2 by operating the moving structure 9. On the other hand, since the irradiated area on the surface of the treatment target organ A changes as time goes, the heat generation of the vicinity of the surface becomes a non-continuous state, and excess temperature rise is prevented in a similar manner.

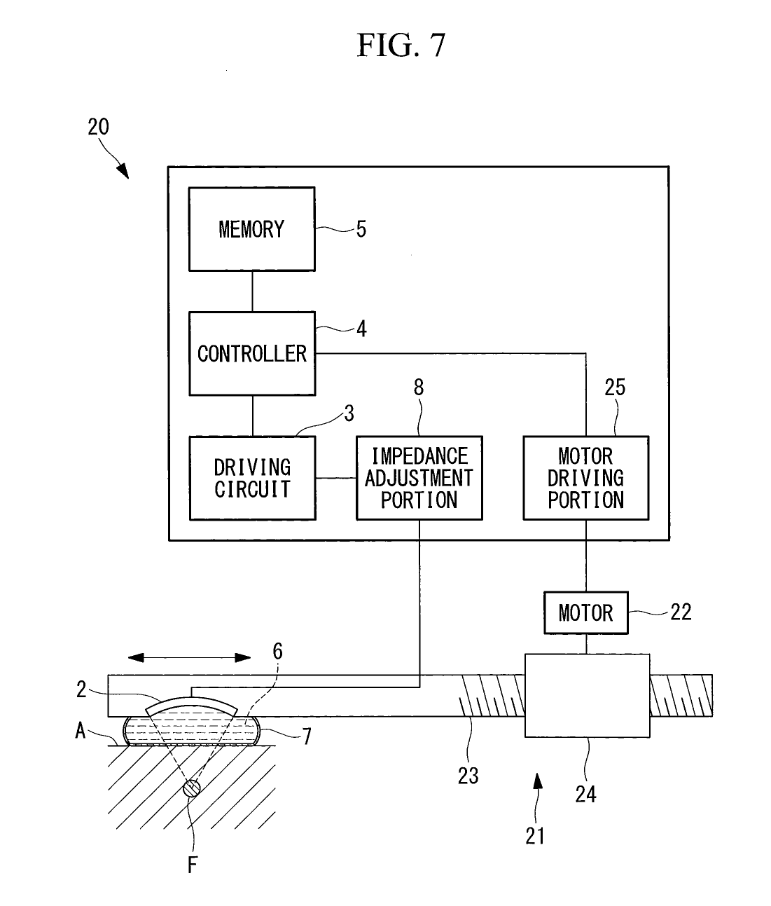

The moving structure 9 which physically moves the ultrasonic element 2 is explained as the moving unit. In contrast, the moving unit can be configured to alternatively actuate the ultrasonic elements 2a, 2b in order to change irradiated area on the surface of the treatment target organ A in a state in which the ultrasonic element 2a and the ultrasonic elements 2b are arranged so that the ultrasonic elements 2a, 2b have the same focal point F as shown in FIGS. 6A and 6B. By employing this configuration, it becomes possible to perform effective treatment by continuously radiating the ultrasonic wave to the affected area positioned at the focal point F, suppressing excess temperature rise on the surface of the treatment target organ A. The number of ultrasonic elements 2a, 2b can be more than three.

Next, An ultrasonic treatment apparatus 20 according to a second embodiment of the present invention will be described below with reference to the drawings.

In the explanation of this embodiment, the elements which are the same as or similar to those of the first embodiment are assigned the same reference symbols, and the explanations thereof are omitted.

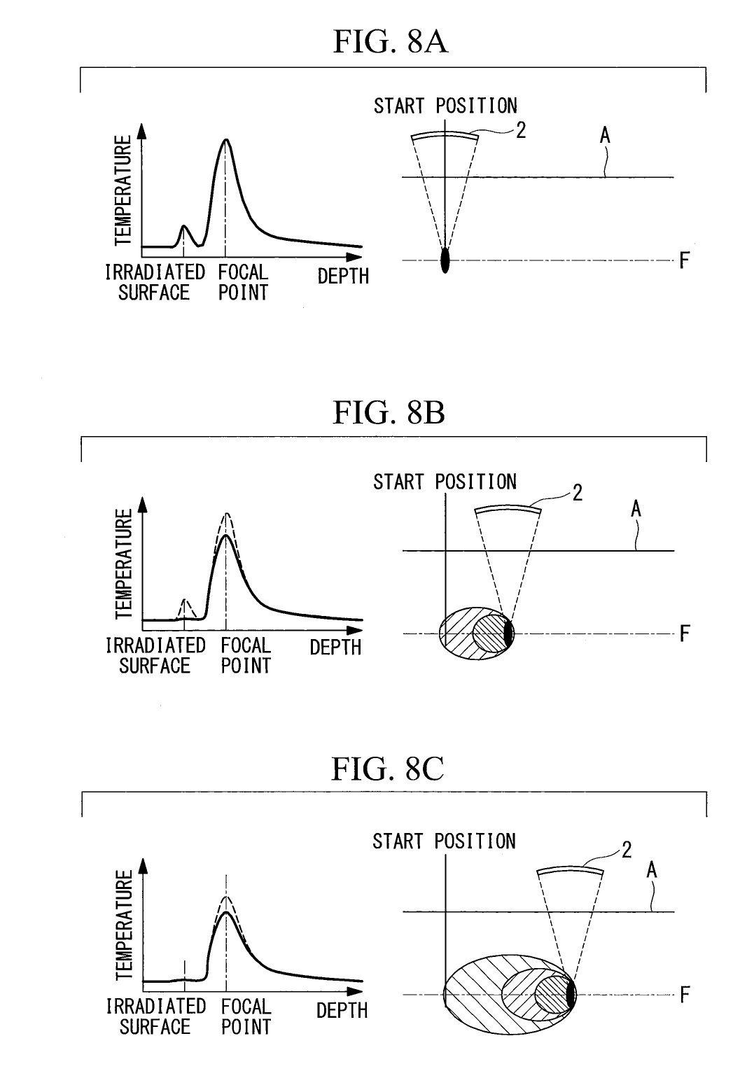

The ultrasonic treatment apparatus 20 according to this embodiment is an apparatus which is not for a state in which there is only one affected area, but for a state in which there are many affected areas scattered around a relatively wide area. As shown in FIG. 7, a moving structure 21 for moving the focal point F of the ultrasonic element is employed.

The moving structure 21 a linear motion mechanism such as a ball screw 23 driven by a motor 22, and moves the ultrasonic element 2 along a straight track. The reference number 24 indicates a nut which is engaged with the ball screw 23 and which is driven by the motor 22, and the reference number 25 indicates a motor driving circuit for driving the motor 22 in accordance with command signals from the controller 4.

When the affected area, which broadly exists, is treated by the ultrasonic treatment apparatus 20 according to this embodiment having the aforementioned configuration, the moving direction of the ultrasonic element 2 is positioned to be parallel to the surface of the treatment target organ A, the ultrasonic element 2 is continuously operated to radiate the ultrasonic wave, and the ultrasonic element 2 is reciprocally moved in the direction along the surface of the treatment target organ A.

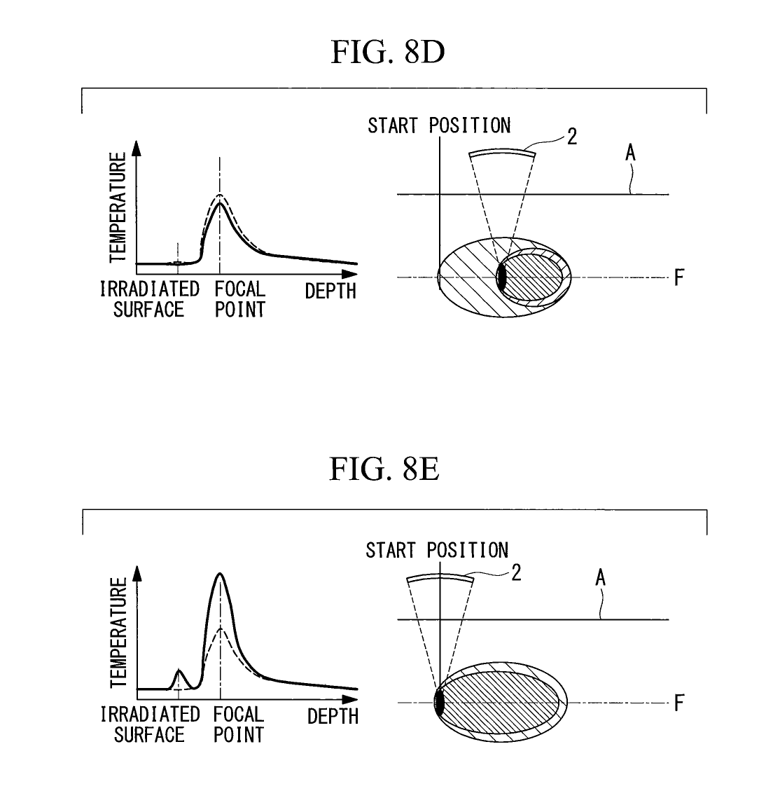

By employing this configuration, as shown in FIG. 8A, when the ultrasonic wave is converged on the focal point F by operating the ultrasonic element 2 at the start position, heat is generated at the focal point F and a vicinity of the surface. However, as shown in FIG. 8B, by moving the ultrasonic element 2 by operating the moving structure 21, the focal point F and the irradiated portion move, and therefore the heated area moves.

Since a large amount of heat is generated at the focal point of the ultrasonic element 2, the heat remains along the moving track of the focal point F when the focal point F moves. On the other hand, since a small amount of heat is generated at the surface, the remaining heat is immediately dispersed and then becomes zero when the irradiated portion of the ultrasonic wave changes as time goes.

Also, as shown in FIGS. 8C and 8D, the high temperature portion is widened over a wide area in the area where the focal point F passes. In the figures, the hatching with narrower spaces shows higher temperature.

As shown in FIG. 8E, when the focal point F returns to the start position, the temperature of the adjacent portion of the focal point F becomes higher than the state of FIG. 8A by heat generation in addition to the remaining heat, but the excess heat rise at the surface is suppressed. By this, it becomes possible to treat the wide affected area positioned at a vicinity of the focal point F, while protecting the vicinity of the surface.

Note that a rack and pinion mechanism or a linear motor can be employed as the linear motion mechanism instead of the ball screw 23.

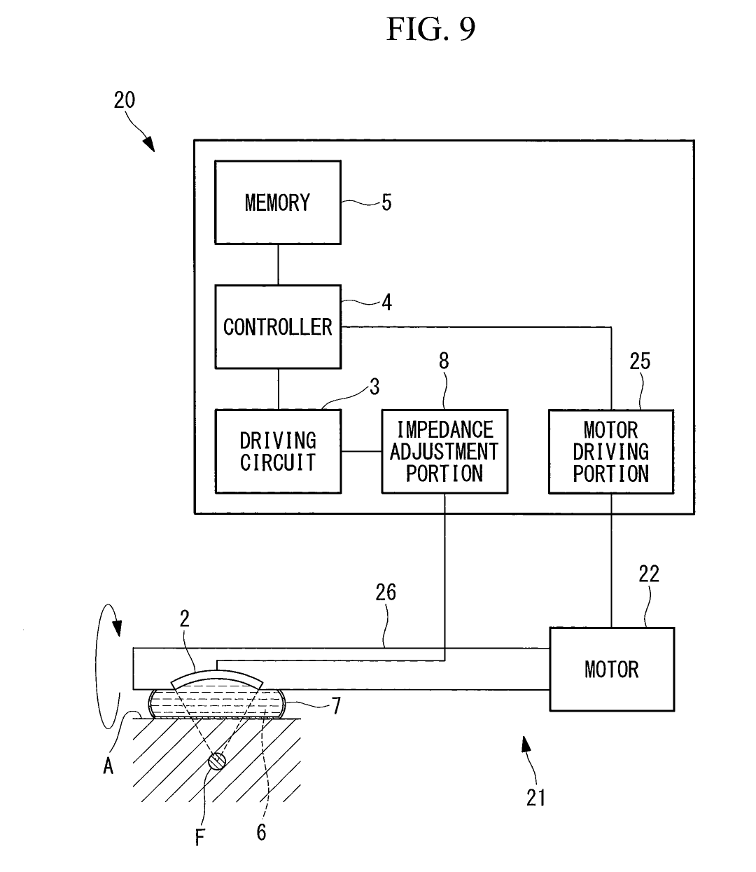

Further, as shown in FIG. 9, a rotation mechanism (motor, for example) 22 which rotates or pivots the ultrasonic element 2 around the longitudinal axis of the inserted portion 26 or an axis which crosses the longitudinal axis can be employed, while the linear motion mechanism has been explained as the moving mechanism 21.

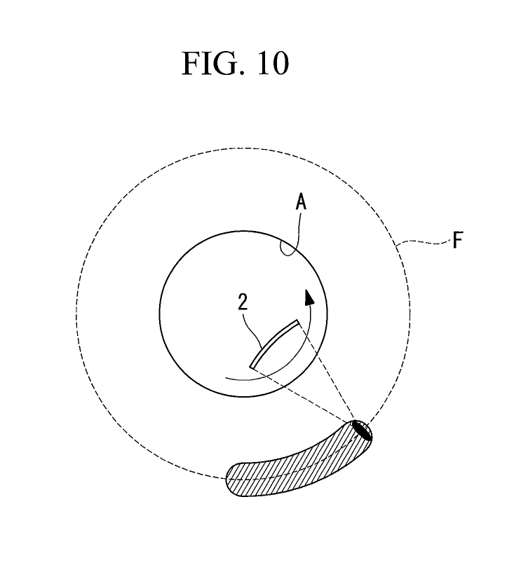

As shown in FIG. 10, when the ultrasonic element 2 is rotated around the longitudinal axis of the inserted portion, the ultrasonic element 2 may be continuously rotated in one direction, and the ultrasonic element 2 is operated within a desired rotational angle area.

Further, a detector (not shown in the figures) such as an encoder may be provided in the moving mechanism 21, and the position and the angle of the ultrasonic element 2 can be accurately adjusted by using feedback control.

In addition, the ultrasonic element 2 and the moving mechanism 21 can be driven in a non-continuous manner instead of the continuous manner.

When the ultrasonic element 2 is moved in a continuous manner, it becomes possible to perform a uniform treatment for a wide area by radiating a certain intensity of ultrasonic wave with increment of the moving speed, or by decreasing the intensity of the ultrasonic wave with uniform speed of movement.

Also, when the ultrasonic element 2 is moved in a non-continuous manner, it is possible to perform a uniform treatment for a wide area by using the following ways: radiating a certain intensity of ultrasonic wave with gradually shortening the duration of radiating the ultrasonic wave in the first state or gradually widening the spaces between the irradiated portions; or gradually lowering the intensity of the ultrasonic wave with a certain space between each pair of the irradiated portions.

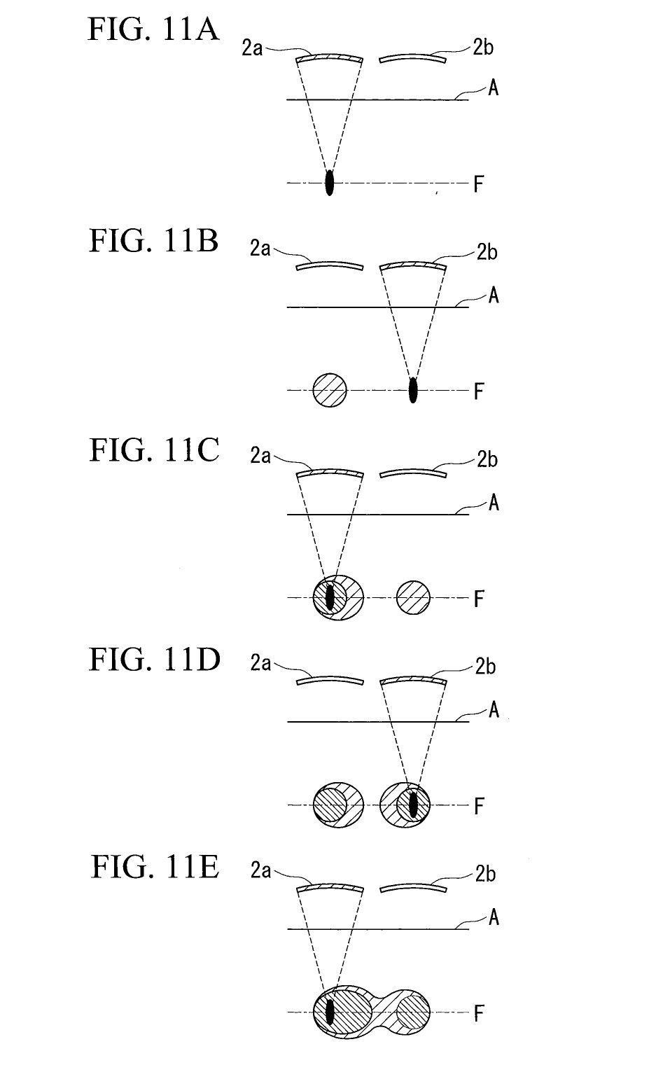

Also, as the moving unit, a configuration in which the irradiated area of the ultrasonic wave is moved by alternatively operating the ultrasonic elements 2a, 2b in a state in which the ultrasonic element 2a and the ultrasonic element 2b are arranged so that the ultrasonic elements 2a, 2b have different focal points F as shown in FIG. 11 may be employed instead of the configuration in which the ultrasonic element 2 is moved. The number of ultrasonic elements 2a, 2b can be more than three.

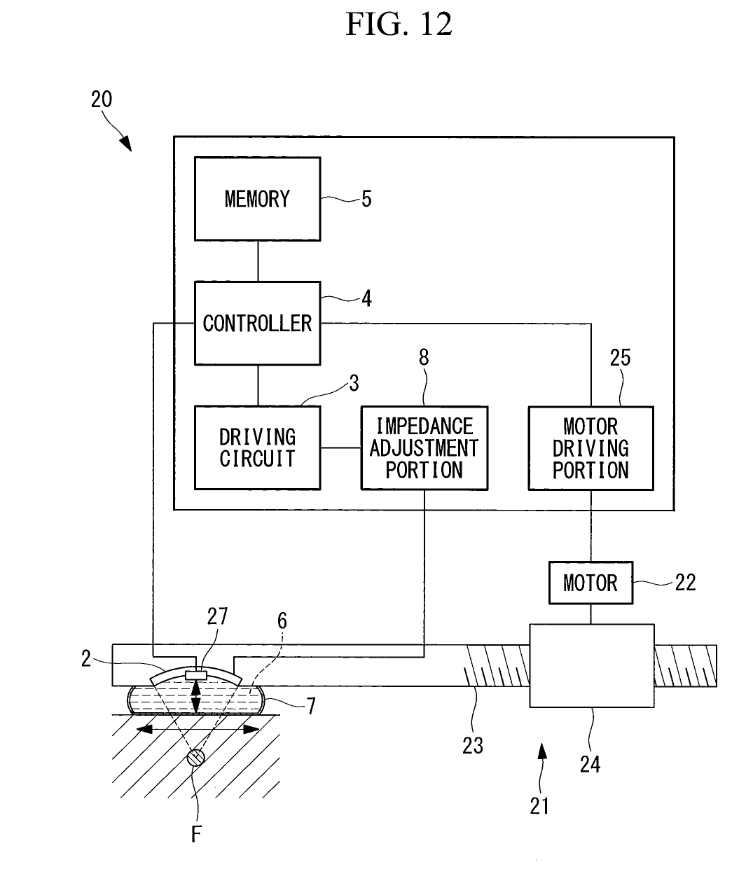

Further, as shown in FIG. 12, a temperature sensor 27 which measures temperature of the irradiated portion of the surface of the treatment target organ A, which is irradiated by the ultrasonic wave from the ultrasonic element 2, may be employed, and the intensity of the ultrasonic and the durations of radiation and non-radiation can be adjusted in accordance with the measured temperature.

By employing this configuration, it becomes possible to more reliably prevent excess temperature rise at the vicinity of the surface of the treatment target organ A.

Further, as the temperature sensor 27, although a non-contact type is preferred, a contact type sensor may be used. When a non-contact type is employed, it is preferable to improve the measuring accuracy by making the measuring position of the temperature sensor 27 constant by employing a relatively stiff balloon which can maintain a constant distance between the ultrasonic element 2 and the treatment target organ A as the balloon 7 in which the acoustic propagation medium 6 is filled.

As the relatively stiff balloon 7, one made of film with low elasticity, one having a skeleton such as a stent, and etc. can be employed.

Also, when the distance between the ultrasonic element 2 and the treatment target organ A is made changeable, it becomes possible to measure temperature of the middle portion of the irradiated area irradiated by the ultrasonic wave regardless of the change of the distance by locating the temperature sensor 27 at the center side of the ultrasonic element 2. In an alternative way, when the temperature sensor 27 is located at a side portion of the ultrasonic element 2, since the temperature of the middle portion of the irradiate area irradiated by the ultrasonic wave is measured from an oblique direction, the measured point is changed when the distance changes.

In this state, it is preferable that a distance sensor (not shown in the drawings) is employed, and that the angle of the temperature sensor 27 is adjusted based on the distance measured by the distance sensor in a configuration in which the angle of the temperature sensor is changeable. With this configuration, when the depth of the affected area varies, it is possible to make the focal position of the ultrasonic element 2 match to the affected area by changing the thickness of the balloon 7 in which the acoustic propagation medium 6 is filled, and also it is possible to accurately control the ultrasonic wave by measuring the temperature of the same position of the irradiated area.

Note that the temperature measuring position is not necessarily located at the center of the irradiated area, and that may be located at the periphery of the irradiated area.

Also, in a case in which the ultrasonic element 2 is moved, and when the diameter of the measuring spot area of the temperature sensor 27 is x[mm], and the necessary time for reaching a threshold temperature is t[s], it is preferable that the moving speed of the element is more than x/t[mm/s]. By this configuration, it becomes possible to continuously radiate the ultrasonic wave so that each portion does not reach the threshold temperature.

Also, although the condition for radiating ultrasonic wave is stored in the memory 5 in this embodiment, it is possible to provide a configuration in which an input portion (not shown in the drawings) is employed and in which the condition for radiating ultrasonic wave can be inputted or selected using the input portion. It is also possible to set a treatment portion or a treatment area using the input portion.

The inventor has arrived at the following aspects of the invention.

An aspect of the present invention provides an ultrasonic treatment apparatus comprising: an ultrasonic element which faces a surface of a treatment target organ through an acoustic propagation medium and which generates ultrasonic wave so that the ultrasonic wave is converged on a depth position of the treatment target organ; and a controller for adjusting the ultrasonic wave irradiated to the treatment target organ from the ultrasonic element, wherein the controller is configured to change intensity of the ultrasonic wave which is irradiated into an area of the surface so that heat which is generated when the ultrasonic wave radiated by the ultrasonic element passes through the surface and which remains at a vicinity of the surface is kept equal to or less than a predetermined threshold value, and the controller performs the intensity change as time passes.

According to this aspect, the controller operates the ultrasonic element and then generates the ultrasonic wave in a state in which the ultrasonic element faces the surface of the treatment target organ through the acoustic propagation medium, the ultrasonic wave is converged on the depth position of the treatment target organ, and therefore the affected area positioned at the depth position of the treatment target organ is heated for treatment. In this state, the ultrasonic wave radiated from the ultrasonic element is irradiated on the surface of the treatment target organ, passing through the acoustic propagation medium, and then the ultrasonic wave also passes through tissue from the surface to the focal point. Therefore, the tissue also generates heat.

Although the heat generated by the ultrasonic wave which has not been converged on the way to the focal point is sufficiently smaller than that generated by converged ultrasonic wave at the focal point, when the ultrasonic wave is continuously irradiated on the same area until the treatment of the affected area positioned at the focal point is completed by heating, heat accumulated at portions other than the focal point. With this aspect, since the intensity of the ultrasonic wave irradiated on the same area of the surface is changed as time passes, as compared with a case in which irradiation is continuous, it becomes possible to make remaining heat at a vicinity of the surface of the treatment target organ equal to or less than a predetermined threshold value. Thus, it is possible to treat the treatment target organ, suppressing temperature rise at the surface, without using a large apparatus which performs, for example, circulation of cooling water as conventionally employed.

In the above-described aspect, it is possible that the controller is configured so that a first state in which the ultrasonic wave is irradiated into the same area of the surface of the treatment target organ and a second state in which the intensity of the ultrasonic wave is lowered relative to the first state in order to lower temperature of the surface raised by the irradiation of the first state is alternatively repeated.

With this configuration, in the first state, the ultrasonic wave radiated from the ultrasonic element is irradiated into the treatment target organ through an area of the surface, and the ultrasonic wave is converged on the affected area positioned at the depth position of the treatment target organ, and then the affected area is treated by heating the affected area by the ultrasonic wave converged on the focal point.

In this state, heat is also generated on the way to the focal point. In the second state, it is possible to suppress heat generation at an area from the surface of the treatment target organ to the focal point by lowering the intensity of the ultrasonic wave irradiated on the same area. On the other hand, heat generation at a vicinity of the focal point is also suppressed in the second state, since heat generation at the focal point during irradiation of the ultrasonic wave is sufficiently larger than heat generation of another portion, remaining heat is large. Therefore, the heated state before the second state is easily regained by the next first state, and therefore continuous treatment of the affected area becomes available.

In the above-described aspect, it is possible that the controller is configured to control the ultrasonic element to change the intensity of the irradiated ultrasonic wave.

By employing this aspect, with the first state and the second state, it becomes possible to treat the affected area at a vicinity of the focal point by heating, while suppressing heat generation at the portion between the surface on which the ultrasonic wave is irradiated and the focal point.

In the above-described aspect, it is possible that the controller is configured to stop radiation of the ultrasonic wave from the ultrasonic element in the second state.

By employing this configuration, heat generation is most efficiently suppressed in the second state by stopping the radiation of the ultrasonic wave.

Also, in the above-described aspect, a moving unit which moves irradiated area on the surface of the treatment target organ on which the ultrasonic wave is irradiated by the ultrasonic element may be employed, and the controller is configured to control the moving unit.

With this configuration, by moving the irradiated area of the ultrasonic wave from the ultrasonic element by using the moving unit controlled by the controller, the first state with radiation of the ultrasonic wave and the second state with non-radiation of the ultrasonic wave are performed at respective different timings when focusing on each area. Thus, it becomes possible to treat the affected area at a vicinity of the focal point by heating, while suppressing heat generation on the way to the focal point in order to protect the portion.

Also, in the above-described aspect, the moving unit may be configured to move the ultrasonic element along the surface of the treatment target organ.

With this configuration, the area on the surface to which the ultrasonic wave, which is toward the treatment target organ, is irradiated is changed by moving the ultrasonic element by the moving unit, and therefore it becomes possible to achieve treatment of the affected area near the focal point by heat generation, and to achieve protection of the portion on the way to the focal point by suppressing heat generation thereof.

Also, in the above-described aspect, the moving unit may be configured to move the ultrasonic element so as not to move a focal point of the ultrasonic wave radiated by the ultrasonic element.

With this configuration, the focal point does not move when the irradiated area of the ultrasonic wave is moved by the moving unit. Thus, since heat generation at the affected area near the focal point is maintained while each area is in the second state, it is possible to efficiently achieve treatment of the affected area near the focal point by heat generation, and to achieve protection of the portion on the way to the focal point by suppressing heat generation thereof.

Also, in the above-described aspect, a plurality of the ultrasonic elements may be arranged so that the ultrasonic elements irradiate respective different areas of the surface of the treatment target organ by the ultrasonic wave, and the moving unit may be configured to change the ultrasonic element which radiates the ultrasonic wave.

With this configuration, when the ultrasonic element which radiates the ultrasonic wave is changed by the moving unit, the surface area of the treatment target organ, which is irradiated by the ultrasonic wave, is changed. Thus, it is possible to achieve treatment of the affected area near the focal point by heat generation, and to achieve protection of the portion on the way to the focal point by suppressing heat generation thereof.

Also, in the above-described aspect, the focal points of the ultrasonic elements may be positioned at a same point.

With this configuration, when the irradiated area of the ultrasonic wave at the surface of the treatment target organ is changed by changing the ultrasonic element which radiates the ultrasonic wave, the focal point of the ultrasonic wave is not changed. Thus, since heat generation at the affected area near the focal point is maintained while each area is in the second state, it is possible to achieve treatment of the affected area near the focal point by heat generation, and to achieve protection of the portion on the way to the focal point by suppressing heat generation thereof.

Further, in the above-described aspect, a temperature sensor which measures temperature of an irradiated area of the ultrasonic wave at the surface of the treatment target organ may be employed, and also the controller is configured to control the ultrasonic wave irradiated on the treatment target organ from the ultrasonic element on the basis of measured temperature by the temperature sensor.

With this configuration, it becomes possible to accurately achieve heat generation at the focal point, and to accurately achieve suppression of heat generation at the portion between the surface and the focal point.

Further, in the above-described aspect, the sensor may be a non-contact type sensor, and also the apparatus can comprise a holding member which keeps distance between the ultrasonic element and the irradiated area at a predetermined distance.

With this configuration, the distance between the ultrasonic element and the irradiated area is kept constant by operation of the holding member, and thereby accurate temperature measurement by the non-contact type sensor becomes available.

Further, in the above-described aspect, the moving unit may be configured to move the ultrasonic element with a speed which is faster than x/t [mm/s] when a diameter of a measured spot area of the sensor is x [mm] and necessary time for reaching a threshold temperature is t [s].

With this configuration, it is possible to continuously radiate the ultrasonic wave in such a situation that each of the portions does not reach the threshold temperature.

The aforementioned aspects can achieve an advantage of enabling treatment of the treatment target organ, suppressing raise of the temperature at the surface, without making the apparatus large.

### REFERENCE SIGNS LIST

A treatment target organ
F focal point
1, 20 ultrasonic treatment apparatus
2, 2a, 2b ultrasonic element
4 controller
6 acoustic propagation medium
7 balloon (holding member)
9, 21 moving structure (moving unit)
27 temperature sensor (sensor)

1. An ultrasonic treatment apparatus comprising:
an ultrasonic element configured to irradiate ultrasonic wave to a treatment target organ through an acoustic propagation medium so that the ultrasonic wave is converged on a depth position of the treatment target organ; and
a controller configured to adjust the ultrasonic wave irradiated to the treatment target organ from the ultrasonic element,
wherein the controller is configured to change intensity per time of the ultrasonic wave which is irradiated into an area of a surface of the treatment target organ so that remaining heat generated at a vicinity of the surface is kept equal to or less than a predetermined threshold value.

2. The ultrasonic treatment apparatus according to claim 1, wherein the controller is configured to switch states of irradiation of the ultrasonic wave between a first state and a second state, wherein
the second state is a state in which an intensity of the ultrasonic wave is lowered than an intensity of the first state of the ultrasonic wave in order to lower temperature of the surface.

3. The ultrasonic treatment apparatus according to claim 2,
wherein the controller is configured to stop irradiation of the ultrasonic wave from the ultrasonic element in the second state.

4. The ultrasonic treatment apparatus according to claim 1, further comprising
a moving unit which moves an irradiated area on the surface of the treatment target organ on which the ultrasonic wave is irradiated by the ultrasonic element,
wherein the controller is configured to control the moving unit.

5. The ultrasonic treatment apparatus according to claim 4,
wherein the moving unit is configured to move the ultrasonic element along the surface of the treatment target organ.

6. The ultrasonic treatment apparatus according to claim 5,
wherein the moving unit is configured to move the ultrasonic element while keeping the focal point of the ultrasonic wave radiated by the ultrasonic element.

7. The ultrasonic treatment apparatus according to claim 4,
wherein the ultrasonic element comprises a plurality of the ultrasonic elements which irradiate different areas of the surface of the treatment target organ by the ultrasonic wave, and
wherein the moving unit is configured to change the ultrasonic element which radiates the ultrasonic wave.

8. The ultrasonic treatment apparatus according to claim 7,
wherein the focal points of the plurality of the ultrasonic elements are positioned at a same point.

9. The ultrasonic treatment apparatus according to claim 1, further comprising
a temperature sensor which measures temperature of an irradiated area of the ultrasonic wave at the surface of the treatment target organ,
wherein the controller is configured to control the ultrasonic wave irradiated to the treatment target organ from the ultrasonic element based on the temperature measured by the temperature sensor.

10. The ultrasonic treatment apparatus according to claim 9,
wherein the temperature sensor is a non-contact type sensor, and
wherein the apparatus further comprises a holding member which keeps distance between the temperature sensor and the irradiated area at a predetermined distance.

11. The ultrasonic treatment apparatus according to claim 5,
wherein the moving unit is configured to move the ultrasonic element with a speed which is faster than x/t [mm/s] when a diameter of a measured spot area of the sensor is x [mm] and necessary time for reaching a threshold temperature is t [s].
